(19) United States
(12) Patent Application Publication
(10) Pub. No.: US 2005/0124898 A1
(43) Pub. Date: Jun. 9, 2005

(54) Method and apparatus for isolating a catheter interface

(75) Inventors: Simcha Borovsky, Fair Lawn, NJ (US); Charles Bryan Byrd, Medford, NJ (US); Praveen Dala-Krishna, Bensalem, PA (US); David Norton, Voorhees, NJ (US)

Correspondence Address: HELLER EHRMAN WHITE & MCAULIFFE LLP, 1717 RHODE ISLAND AVE, NW, WASHINGTON, DC, 20036-3001, US

(73) Assignee: EP MedSystems, Inc., West Berlin, NJ (US)

(21) Appl. No.: 10/997,898

(22) Filed: Nov. 29, 2004

Related U.S. Application Data
(63) Continuation-in-part of application No. 10/345,806, filed on Jan. 16, 2003.
(60) Provisional application No. 60/349,060, filed on Jan. 16, 2002.

(51) Int. Cl. A61B 8/14

(52) U.S. Cl. 600/467

(57) ABSTRACT

An interface for limiting an amount of current passing to an intra-body medical device is provided, the interface including a first catheter port configured for coupling with a first catheter, a first processor port configured for coupling with a first cable linkable to a first processor, and a current isolator coupled to the first catheter port and to the first processor port, the current isolator limiting an amount of current passing to the first catheter port.

### CORRESPONDING RELATED APPLICATIONS

The present invention is a continuation in part. (CIP) of parent application Ser. No. 10/345,806 entitled “ULTRASOUND IMAGING CATHETER ISOLATION SYSTEM WITH TEMPERATURE SENSOR” filed on Jan. 16, 2003, claiming the benefit of U.S. Provisional Patent Application Ser. No. 60/349,060, filed on Jan. 16, 2002. The present application claims the benefit of and priority to these applications, the entire contents of which being incorporated by reference herein in their entirety.

This application is also related to co-pending application ______. entitled “SAFETY SYSTEMS AND METHODS FOR ENSURING SAFE USE OF INTRA-CARDIAC ULTRASOUND CATHETERS”, filed concurrently herewith. The entire contents of this co-pending application are incorporated by reference herein in its entirety.

### BACKGROUND OF THE INVENTION

1. Field of the Invention

The present invention is directed toward improvements in ultrasonic=imaging catheters and more particularly toward a current isolation set-up useable in conjunction with an ultrasound machine that allows a connecting mechanism be placed easily on or near a patient and connected to an ultrasound machine by a reusable connector cable.

2. Description of the Related Art

Medical imaging technology is used to improve the diagnosis and treatment of medical conditions. Presently available medical imaging technology includes a wide variety of ultrasound, X-ray, nuclear, magnetic resonance imaging (MRI) and other imaging systems.

For some medical imaging technologies, such as those involving intra-body probes (e.g., ultrasound imaging catheters, electrophysiology (EP) catheters, ablation catheters, etc.), particular attention is paid to electrical safety concerns arising from use of electrical devices within a patient's body. By way of example, for intra-cardiac ultrasound catheters, testing has shown that leakage currents of sufficient strength can cause muscle stimulation which may be detrimental to the patient undergoing intra-body imaging. As such, industry approved electrical safety standards (e.g., for isolation, grounding, and leakage current) have been established for medical devices, such as national standards set by the Association for Advancement of Medical Instrumentation, limiting leakage currents from intracardiac probes to less than 50 μ amps.

In some conventional devices, such as catheter based probes, shielding is provided by way of a sturdy catheter body to satisfy the industry approved electrical safety standards. Shielding alone, however, may be unsatisfactory for some implementations, as substantial shielding increases the thickness of the catheter body. Induced currents may also arise from the catheters acting as an antenna picking up energy radiated by electronic equipment present in a typical electrophysiology lab. Further, in some instances the shielding may become inadvertently damaged and thus not provide adequate protection. As such, a need exists for improved methods and devices that meet or exceed the industry approved electrical safety standards for medical devices.

Recently published research has revealed that the human heart is more vulnerable to small currents when introduced within the heart itself, such as by percutaneous catheters. In CARDIOVASCULAR COLLAPSE CAUSED BY ELECTROCARDIOGRAPHICALLY SILENT 60-HZ INTRACARDIAC LEAKAGE CURRENT, C. Swerdlow et. al., which is incorporated by reference herein in its entirety, it is reported that leakage currents as low as 20 μ amps may induce cardiovascular collapse when applied within the heart. Accordingly, percutaneous catheters might require greater electrical isolation than specified in current industry standards to assure patient safety.

Another problem with conventional devices, especially with multi element arrayed ultrasound catheters, is that the cabling from the ultrasound machine to the catheter, and from the catheter proximal connector to the catheter transducer housed at the distal tip, is expensive. A first solution to keep this expense low, is to move the ultrasound machine next to the bed. This is impractical, as most catheter rooms are sterile or semi-sterile environments, and the machine may have to be maintained some distance from the patient bedside. Thus, a connecting cable which is reusable (and probably non-sterile) is desirable, as opposed to the catheter itself, which is sterile and usually not re-usable. It would be most desirable if this connecting cable could be used as a universal cable in that it could be used with many ultrasound machines. Such an isolation mechanism might also be used to connect multiple equipment to the patient, such as in an electrophysiology (EP) study wherein a recording system, a mapping system, and an ultrasound system could all be used simultaneously on the patient Many ultrasound machines have a standard 200 pin zero insertion force (ZIF) connector, but most ultrasound machines do not have patient isolation built in to the degree necessary for percutaneous catheter use.

Other problems with the prior art not described above can also be overcome using the teachings of the present invention, as would be readily apparent to one of ordinary skill in the art after reading this disclosure.

### SUMMARY OF THE INVENTION

According to an embodiment of the present invention, an interface for limiting the amount of current passing to an intra-body medical device is provided, the interface including a first catheter port configured for coupling with a first catheter, a first processor port configured for coupling with a first cable linkable to a first processor, and a current isolator coupled to the first catheter port and to the first processor port, the current isolator limiting the amount of current passing to the first catheter port. Preferably, the current isolator limits the maximum amount of current that can be passed from system ground to the first catheter port (or vice-versa) to not more than about 50 μA. More preferably, the current isolator limits the maximum amount of current passing to the first catheter port from system ground (or vice-versa) to not more than about 25 μA. Most preferably, the current isolator limits the amount of current passing to the first catheter port from the system ground (or vice-versa) to not more than about 20 μA.

According to another embodiment of the present invention, a method of limiting an amount of current that can-be passed from the system ground to the ground plane of an intra-body medical device is provided, the method including receiving, at an isolation box, an imaging signal from a first catheter, isolating this received signal electrically from the imaging system, with the isolation circuit allowing not more than 25 μA to leak through, and sending the imaging signal from the first catheter to a first processor with zero or minimal attenuation.

According to another embodiment of the present invention, an isolation/junction box for a medical imaging system is provided, including a first port for coupling with an imaging ultrasound catheter, a plurality of second ports for coupling with a plurality of second catheters of a different type than the imaging ultrasound catheter, a third port for coupling with a first imaging workstation cable, and isolation circuitry for isolating current(s) passing from the third port to at least one of the first port and the plurality of second ports to not more than about 25 μA.

### BRIEF DESCRIPTION OF THE DRAWINGS

FIG. 1 is a representation of a facility in which a patient undergoes intra-body imaging according to an embodiment of the present invention;

FIG. 2 is a representation of a catheter employing an embodiment of the present invention with an isolation system shown schematically;

FIG. 3 is a detailed view of an end of the catheter shown in FIG. 2 illustrating an ultrasound transducer and a thermistor;

FIG. 4 is a schematic representation of an isolation circuit according to an embodiment of the present invention;

FIG. 5 is a schematic representation of an isolation box according to an embodiment of the present invention;

FIG. 6 is a schematic view of linear phased array transducer with a thermistor according to an embodiment of the present invention; and

FIG. 7 is a perspective view of an isolation box according to an embodiment of the present invention.

### DETAILED DESCRIPTION OF THE EXEMPLARY EMBODIMENTS

Reference will now be made in detail to exemplary embodiments of the present invention. Wherever possible, the same reference numbers will be used throughout the drawings to refer to the same or like parts.

The present invention provides improved electrical isolation for catheters and similar probes that may be inserted in a body, such as the body of a mammal, including a human. Electrical isolation is important because leakage of electrical current may lead to deleterious effects, including cardiac arrhythmia or cardiovascular collapse. Two types of isolation are important. One type, sometimes referred to as “patient sink leakage current,” may arise when an external source of voltage, such as 250 volts from an electrical outlet or electrical equipment, passes through the catheter and the patient's body or between one catheter element and another catheter or catheter element within the body. This type of isolation is associated with equipment fault conditions and is addressed in the design of intrabody probe systems (e.g., electrophysiology electrode catheters and ultrasound imaging catheters) to protect the patient from deleterious shocks caused by an electrical fault. A second type of isolation limits leakage currents that may arise under no fault conditions. Long probes containing conductors, such as electrophysiology and ultrasound imaging catheters, may exhibit electric currents induced in the conductors by electromagnetic radiation present in the room. The longer the electrical leads, the greater the potential induced current. Patient safety requires limiting both types of leakage currents to low (e.g., 20 microamps or less) levels. Generally, fault-type leakage currents may be isolated on a per catheter (or probe) basis, since a single failure presents a significant threat and there is a low likelihood that multiple faults will occur simultaneously. In contrast, no fault leakage isolation must address multiple catheters (or probes), since leakage currents from induced currents in multiple catheters may be additive. Consequently, isolating each catheter provides improved patient safety. Providing such isolation dose to the patient so as to reduce the length of electrical conductors on the patient side of the isolation also improves patient safety. Such measures are of particular importance for intracardiac probes.

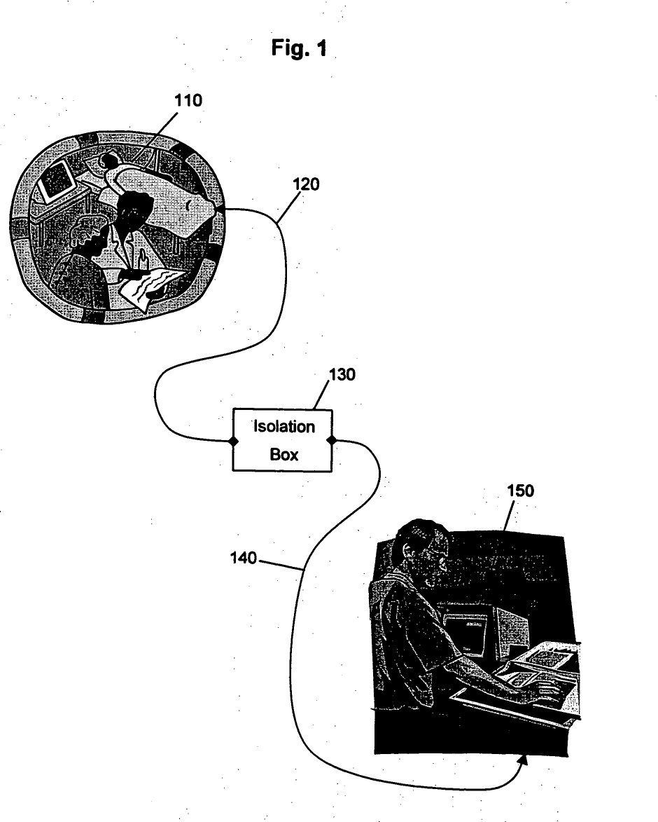

According to an embodiment of the present invention as shown in FIG. 1, an isolation box 130 is provided that electrically couples an imaging probe 120 to an ultrasound equipment 150 via a cable 140. The isolation box 130 is positioned as dose to the patient 110 as possible consistent with room layout and sterility limitations (e.g., attached to the patient's bed or operating/imaging table) so as to minimize the length of imaging probe 120.

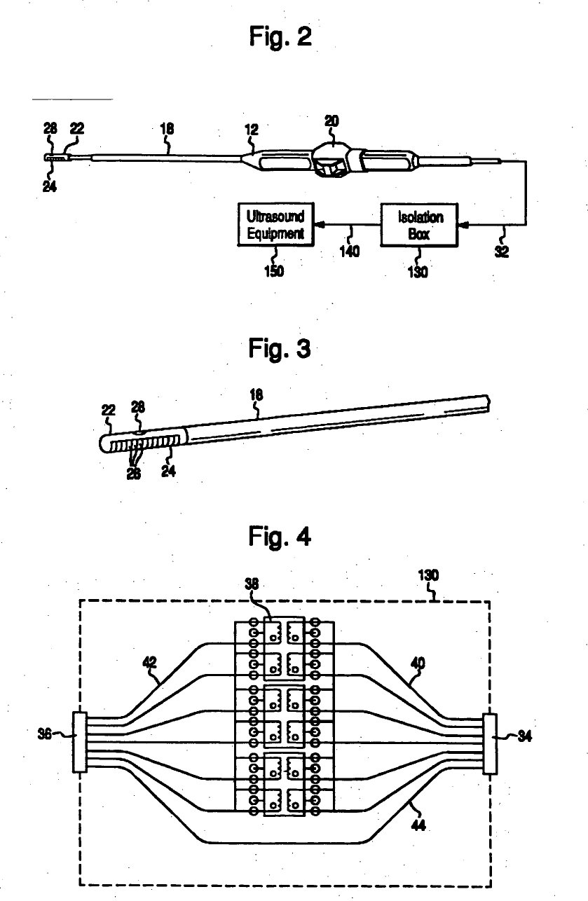

The imaging probe 120 preferably includes a catheter assembly 12 as shown in FIG. 2. In many respects, the catheter assembly 12 may be constructed in a conventional manner similar, for example, to the ultrasound imaging catheter described in application Ser. No. 09/263,755 filed Mar. 5, 1999, the entire content of said application being incorporated herein by reference. It should be noted that the present invention is not limited to the specific catheter assembly disclosed in the prior referenced application as the invention is applicable to various catheters designed for intravascular/intracardiac echocardiography and for other physiological uses. In particular, the present invention is applicable to any intravascular/intracardiac catheter including an electrical conductor (e.g., a signal or guide wire) that carries electrical currents or in which electrical currents may be induced by electromagnetic radiation.

The catheter assembly 12 includes an elongated catheter generally in the form of a tube 18. The proximal end of the tube 18 is connected to a handle mechanism 20 which could include means for controlling the steering of an ultrasound probe 22 mounted at the distal end of the catheter tube 18. The ultrasound probe 22 includes an ultrasound transducer assembly 24, which is comprised of a number of ultrasonic transducer elements 26 having wires connected thereto which are provided inside the tube. Although only twelve or so transducer elements 26 are shown in FIG. 3, substantially any number of transducer elements may be employed as described in the prior application discussed above. These transducer elements can also be deployed in many geometrical orientations, such as linear, circular, curved, etc. Typically, the transducer includes sixty-four elements.

Mounted near the distal end, such as on the reverse side of the ultrasound transducer probe 22 is a thermistor 28. The thermistor 28 is preferably embedded within the probe 22 so as to provide a smooth outer surface on the probe 22. The exact location of the thermistor 28 is not critical. However, it must be in such a position so as to be able to sense the temperature of the tissue in the vicinity of the probe 22 and/or the temperature of the probe 22 itself without interfering with the operation of the same. Furthermore, while the invention has been described with specific reference to a thermistor 28, it should be readily apparent that other types of safety-related sensors may also be employed which are capable of sensing temperature or other safety-related parameters. The electrical wires leading from the thermistor 28 pass through the inside of the catheter tube 18 to the exterior of the body in substantially the same manner as the numerous wires connected to the ultrasonic transducer elements 26.

The ultrasonic equipment 150 illustrated in FIG. 1 is a conventional ultrasound machine which may operate in a manner well known in the art. This equipment 150 may be located a distance from a patients bed outside of the sterile area. The isolation box 130, however, is intended to be placed on or near a patients bed within the sterile area, with cable 140 connecting the two together. Alternatively, the isolation box 130 may be placed at the boundary of the sterile area as described more fully below.

The cable 32 from the catheter assembly 12 carries a plug at the end thereof that plugs into the isolation box 130 to form the various electrical connections. Since the isolation box 130 is relatively small and is located on or near the patients bed, the cable 32 can also be relatively short, thereby reducing the cost of the same. The cable 32 carries all of the leads from the ultrasonic transducers 26, the leads from the thermistor 28 and any other leads that may be used in connection with the catheter assembly 12. For example, the catheter assembly 12 may carry other electrodes and/or transducers at or near the tip thereof or elsewhere along the catheter body 18 for various other purposes..

The isolation box 130 preferably has an input connector or socket 34 for connection to the cable 32 and an output socket or connector 36 for connection to the cable 140 that leads to the ultrasound equipment 150. These may be the card connector disclosed herein or conventional sockets or connectors well known in the art.

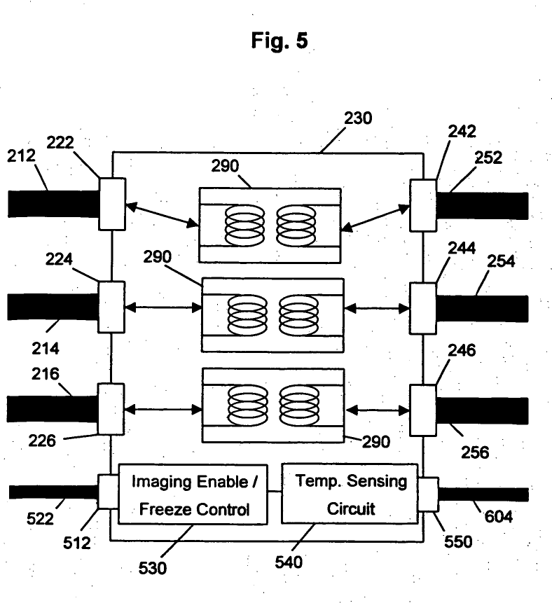

According to an embodiment of the present invention, the isolation box 130 includes a plurality of isolation transformers 38 as shown in FIG. 4. In order to keep the isolation box 130 as small as possible, relatively small transformers 38 are utilized as a significant number of them are typically implemented within the isolation box 130. There may be one isolation transformer 38 for each of the ultrasonic transducer elements 26 and, potentially, the thermistor 28. Thus, If there are sixty-four transducer elements 26, an equal number (i.e., sixty-four transformers) of transformers 38 are required. Suitable transformers provide for transmission of the received signal with zero or minimal attenuation. One transformer which may be used with various embodiments of the present invention is a wide band isolation transformer sold by Rhombus Industries Inc. of Huntington, Calif. as Rhombus Model No. T-1113.

One side of each transformer 38 may be connected by leads 40 to the socket or connector 34 so as to be connected to the transducer assembly 12 by way of the cable 32. Similar leads 42 connect the opposite side of each transformer 38 to the socket or connector 36 for ultimate connection to the ultrasound equipment 150 through the cable 140. Other leads such as shown at 44 may pass directly through the isolation box 130 from connector 34 to connector 36 without being connected to an isolation transformer 38 if the same is desired. For example, the lead from the thermistor 28 may or may not pass through an isolation transformer 38 but may be connected directly to the ultrasound equipment 150 by passing through the isolation box 130 with appropriate opto-isolator circuits located in the isolation box. Alternative isolation circuitry may also be employed. As should be readily apparent to those skilled in the art, appropriate circuitry may be located either in the isolation box 130 or the ultrasound equipment 150 or elsewhere for interpreting the signal from the thermistor 28 for controlling the ultrasound equipment 150 in response thereto.

An isolation box 230 according to an embodiment of the present invention is shown in the block diagram of FIG. 5. According to this embodiment, the isolation box 230 includes a plurality of probe ports 222, 224, 226 for coupling to a plurality of probe elements 212, 214, 216 respectively. While only three probe ports 222, 224, 226 and three probe elements 212, 214, 216 are shown, the number of ports and probes, and the types of ports and probes may vary for any given implementation. By way of example, the isolation box 230 may include sixty-four or more contact edge connector type ports for sixty-four individual probe elements. Further, the same isolation box 230 might also include one or more other connectors that pertain to other elements, such as an EP recording system, a mapping system, etc., as shown in the exemplary implementation of FIG. 7.

The probe ports 222, 224, 226 are coupled to one or more processor ports 242, 244, 246 (after passing through isolation circuit 290) for coupling to a corresponding number of processor cables 252, 254, 256. According to one embodiment of the present invention, the processor ports are integrated into a one or more high-density ZIF connector(s) 710 as shown in FIG. 7. The integrated port 710 may be coupled to one or more processors via one or more high density cables. Other configurations are also contemplated.

Additionally, one ore more of ports 242, 244, 246 and/or 710 may be configured to have a card connector pass through a plastic barrier to establish an electrical connection therewith. In this manner, the plastic barrier serves as a boundary between the sterile and non-sterile environments, and may be disposable to allow re-use of one or more of the various components. The plastic barrier may comprise, for example, a plastic sleeve/bag, etc.

According to an embodiment of the present invention, filter(s) may be included in the isolation box to suppress noise on an imaging signal from a probe of a first type caused by a probe of a second type. As an example, a bandpass filter may be employed in-line with an ultrasound imaging probe element to suppress noise generated by a radio-frequency (RF) probe. By providing signal filtering such as band limiting filters, the isolation box provides greater capacity for multiple probes of differing types to operate at the same time. Similarly, stages of amplification and impedance matching circuits could also be deployed to enhance signal-to-noise ratios of various signals passed through such an isolation mechanism.

Additionally, according to an embodiment of the present invention, the thermistor 28 automatically shuts off the catheter assembly 12 at the isolation box 130. By way of example, an output of thermistor 28 may be coupled to an enable/disable input to a plurality of gates gating wires passing to/from the transducer elements 26. So long as the temperature of catheter assembly 12 remains below a safe level (e.g., not more than 43° C.), the gates remain enabled allowing signals to pass to/from transducer elements 26. However, should the temperature of catheter assembly 12 reach or exceed an unsafe level, thermistor 28 disables the gates, automatically shutting off the catheter assembly 12.. Other configurations for automatic shutoff are also contemplated.

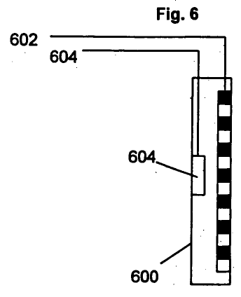

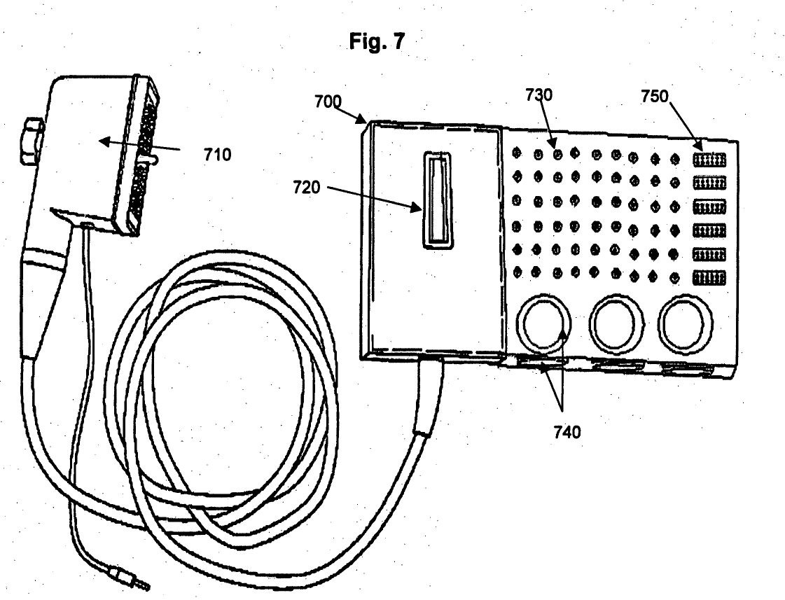

One such scenario would be to provide a thermistor 604 behind a linear ultrasound transducer array 601 (forming part of probe 120), as shown in FIG. 6, coupled via wires 602, 604 to the isolation box 230 shown in FIG. 5. Preferably, the wire 604 is coupled to port 550 on isolation box 230. Additionally, the isolation box 230 includes port 512 coupled to ultrasound equipment 150 via wire 522. The isolation box 230 is configured to disable transmission of ultrasound signals from the ultrasound equipment 150 by disabling the transmit circuitry by signaling the ultrasound equipment 150 through a trigger mechanism such as a hardware interrupt. In particular, the isolation box 230 may include a temperature sensing circuit 540 for sensing a temperature of transducer array 601 via thermistor 604, and an imaging enable/freeze control circuit 530 for disabling the transmit circuitry based on the temperature sensed by temperature sensing circuit 540. Other mechanisms could include disabling an array of multiplexers or transmit channel amplifiers commonly used in such circuits.

An example of an ultrasound catheter connector and isolation system employing an embodiment of the present invention is shown in FIG. 7. In particular, the system includes a patient side isolation module 700 (preferably with thermal cutoff circuitry), with a plurality of interconnection ports for coupling with various electrical components.. In particular, the system preferably includes a ZIF connector 710, a plurality of catheter ports 730, 750, a plurality of position management ports 740, and a card edge connector 720. The ZIF connector 710 may be provided for coupling with ultrasound equipment 150. The plurality of catheter ports 730, 750 may be provided for coupling with intra-body catheters (e.g., ultrasound imaging catheters, electrophysiology catheters, etc.). The plurality of position management ports 740 may be provided for coupling with positioning catheters. Other configurations are also contemplated.

According to an embodiment of the present invention, the current isolator limits the maximum amount of current that can be passed from system ground to the first catheter port (or vice-versa) to not more than about 50 μA. More preferably, the current isolator limits the maximum amount of current passing to the first catheter port from system ground (or vice-versa) to not more than about 25 μA. Most preferably, the current isolator limits the amount of current passing to the first catheter port from the system ground (or vice-versa) to not more than about 20 μA.

According to another embodiment of the present invention, a method of limiting an amount of current that can be passed from the system ground to the ground plane of an intra-body medical device is provided, the method including receiving, at an isolation box, an imaging signal from a first catheter, isolating this received signal electrically from the imaging system, with the isolation circuit allowing not more than 25 μA to leak through, and sending the imaging signal from the first catheter to a first processor with zero or minimal attenuation.

The aforementioned system provides the user with a relatively small, and compact device which can be positioned dose to the patient, and is relatively easy to sterilize. Thus, the system is easier to use, safer for the patient, and has a lower maintenance cost due to a reduction in the amount of single use cabling. Other advantages and features will be readily apparent to those of skill in the art after reading this disclosure.

The foregoing description of various embodiments of the invention has been presented for purposes of illustration and description. It is not intended to be exhaustive or to limit the invention to the precise form disclosed, and modifications and variations are possible in light of the above teachings or may be acquired from practice of the invention. By way of example, the present invention is applicable to any catheter-instrument, such as lasers, optical imagers, thermal ablation devices, RF ablation devices, and ultrasound ablation devices in addition to the devices described above. The embodiments were chosen and described in order to explain the principles of the invention and its practical application to enable one skilled in the art to utilize the invention in various embodiments and with various modifications as are suited to the particular use contemplated.

1. An interface for limiting an amount of current passing to an intra-body medical device, the interface comprising:
a first catheter port configured for coupling with a first catheter, a first processor port configured for coupling with a first cable linkable to a first processor, and
a current isolator coupled to the first catheter port and to the first processor port, the current isolator limiting an amount of current passing to the first catheter port.

2. The interface of claim 1, wherein the current isolator limits the amount of current passing to the first catheter port to not more than about 50 μA in a fault condition.

3. The interface of claim 2, wherein the current isolator limits the amount of current passing to the first catheter port to not more than about 25 μA in a fault condition.

4. The interface of claim 3, wherein the current isolator limits the amount of current passing to the first catheter port to not more than about 20 μA in a no fault condition.

5. The interface of claim 1, further comprising:.
a plurality of second catheter ports for coupling with a corresponding number of second catheters,
wherein the current isolator couples the plurality of second catheter ports to the first processor port, and
wherein the current isolator limits an amount of current passing to at least one of the plurality of second catheter ports.

6. The interface of claim 5, wherein the first processor port comprises a high-density Zero-Insertion-Force (ZIF) connector.

7. The interface of claim 5, wherein at least one of the second catheters is of a different type than the first catheter.

8. The interface of claim 7, further comprising at least one band limiting filter for filtering noise interfering on a signal from the first imaging catheter caused by the at least one second catheter of a different type.

9. The interface of claim 1, further comprising:
a second catheter port for coupling with a second catheter, and
a second processor port for coupling with a second cable linkable to a second processor,.
wherein the current isolator couples the second catheter port to the second processor port, and
wherein the current isolator limits an amount of current passing to the second catheter port.

10. The interface of claim 9, wherein the second catheter is of a different type than the first catheter.

11. The interface of claim 10, further comprising at least one band limiting filter for filtering noise interfering on a signal from the first catheter caused by the second catheter.

12. The interface of claim 1, wherein the first catheter comprises an imaging catheter.

13. The interface of claim 12, wherein the imaging catheter includes at least one ultrasound transducer.

14. The interface of claim 13, wherein the imaging catheter includes a phased array transducer.

15. A medical imaging system including the interface of claim 1.

16. A method of limiting an amount of current passing to an intra-body medical device, comprising:
receiving, at an isolation box, an imaging signal from a first catheter,
isolating an amount of current passing to the first catheter to not more than 50 μA in a fault condition; and
sending the imaging signal from the first catheter to a first processor.

17. The method of claim 16, further comprising:
receiving, at the isolation box, signals from a plurality of second catheters;
isolating current(s) passing to the plurality of second catheters; and
sending the signals from the plurality of second catheters to the first processor.

18. The method of claim 17, wherein at least one of the second catheters is of a different type than the first catheter.

19. The method of claim 18, further comprising filtering noise interfering on an imaging signal from the first catheter caused by the at least one second catheter of a different type.

20. The method of claim 16, further comprising:
receiving a signal from a second catheter,
isolating current passing to the second catheter, and
sending the signal from the second catheter to a second processor.

21. The method of claim 20, wherein the second catheter is of a different type than the first catheter.

22. The method of claim 21, further comprising filtering noise interfering on an imaging signal from the first catheter caused by the second catheter.

23. The method of claim 16, further comprising inserting a push card connector coupled to the first catheter through a sterile boundary barrier to couple with the isolation box.

24. An isolation/junction box for a medical imaging system, comprising:
a first port for coupling with an imaging ultrasound catheter,
a plurality of second ports for coupling with a plurality of second catheters of a different type than the imaging ultrasound catheter,
a third port for coupling with a first imaging workstation cable; and
isolation circuitry for isolating current(s) passing from the third port to at least one of the first port and the plurality of second ports to not more than about 25 μA.

25. The isolation/junction box of claim 24, further comprising:
a fourth port for coupling with a second imaging workstation cable,
wherein the first port is coupled to the third port via the isolation circuitry,
wherein the plurality of second ports are coupled to the fourth port via the isolation circuitry, and
wherein the isolation circuitry isolates current(s) passing from the fourth port to the plurality of second ports to not more than about 25 μA.

26. The isolation/junction box of claim 25, wherein both the first port and the plurality of second ports are coupled to the third port via the isolation circuitry.
